(19) United States
(12) Patent Application Publication
(10) Pub. No.: US 2025/0092423 A1
(43) Pub. Date: Mar. 20, 2025

(54) NANOPARTICLE NUCLEIC ACID CARRIER CONTAINING HYPERBRANCHED POLYLYSINE AND USE THEREOF

(71) Applicant: ZHEJIANG UNIVERSITY, Hangzhou (CN)

(72) Inventors: Changyou GAO, Hangzhou (CN); Qiaoxuan WANG, Hangzhou (CN); Pai PENG, Hangzhou (CN)

(21) Appl. No.: 18/963,700

(22) Filed: Nov. 28, 2024

Related U.S. Application Data
(63) Continuation of application No. PCT/CN2022/097159, filed on Jun. 6, 2022.

(30) Foreign Application Priority Data
May 30, 2022 (CN) 202210600677.3

(51) Int. Cl. C12N 15/87; A61K 9/51; A61K 48/00

(52) U.S. Cl. CPC C12N 15/87; A61K 9/5146; A61K 9/5192; A61K 48/0041

(57) ABSTRACT

A nanoparticle nucleic acid carrier containing hyperbranched polylysine (HBPL) and use thereof provided, which relate to the field of biomaterials. The nucleic acid carrier can be bonded with nucleic acid substances through an electrostatic action to form stable nanoparticles, and the preparation method and application means are both convenient and easy to implement, and the repeatability is high. The nanoparticle nucleic acid carrier containing hyperbranched polylysine provided by the present disclosure has a lower cytotoxicity, can successfully load nucleic acid substances into cells, and obtain a higher transfection rate; moreover, compared with the existing optimal gene transfection carrier, polyethyleneimine (PEI), the nucleic acid carrier can show a higher transfection rate and a lower cytotoxicity, and has good application prospects in the fields of gene therapy, nucleic acid vaccines and the like.

### CROSS-REFERENCE TO RELATED APPLICATIONS

The present application is a National Stage of International Application No. PCT/CN2022/097159 filed Jun. 6, 2022, which claims a priority to Chinese Patent Application No. 2022106006773, filed on May 30, 2022, both of which are hereby incorporated by reference in their entireties.

### TECHNICAL FIELD

The present disclosure relates to a nanoparticle nucleic acid carrier containing hyperbranched polylysine (HBPL) and use thereof, belonging to the field of biomaterials.

### BACKGROUND

Gene therapy is a method of inputting nucleic acid as a medicine into target cells for disease treatment. In recent years, the object of gene therapy is no longer limited to single-gene genetic diseases, and its use scope has gradually expanded to malignant tumors, cardiovascular diseases, autoimmune diseases, genetic engineering vaccines and the like.

In gene therapy, due to a large number of negative charges on the surface of a naked nucleic acid, it is difficult to enter the cells through the same negatively charged cell membrane. In addition, the naked nucleic acid is likely to be degraded by nucleic acid degrading enzymes when transported in vivo. Therefore, it is of great significance for gene therapy to use safe and effective carriers to safely “transport” nucleic acid substances into cells to play their roles.

In previous studies, viruses are often used as carriers to effectively promote nucleic acid transport because of their natural characteristics of infecting host cells. However, the virus carrier still has high cytotoxicity and immunogenicity, which can easily trigger the inflammatory reaction of the body and cause secondary damage to the tissue, and there are problems such as high cost and limited amount of loaded nucleic acid.

Nanoparticles refer to granular dispersions or solid particles with a particle size in the range of 10-1000 nm. Enhanced high permeability and long retention effect can be obtained by using nanoparticles for delivery. Because of its high potential and specific surface area, the nanoparticle carrier has a large nucleic acid load, and has the function of protecting nucleic acid molecules from enzymatic degradation and immunological recognition, and the transport efficiency across cell membrane is higher than other carriers. In addition, nanocarriers prepared from biomaterials usually have good biocompatibility and biodegradability, and have little influence on cell growth and metabolism.

Some cationic polymers have been found to be useful for gene delivery because of their rich sources, diverse structures, easy modification and functionalization. For example, polyethyleneimine (PEI) can be transferred from the endosome to the cytoplasm by its powerful proton sponge effect, but its biological toxicity is high, which limits the use of PEI in gene transfection. Poly(dimethylaminoethyl methacrylate) (PDMAEMA) is also often used in gene delivery research. However, due to a large number of quaternary amines in its structure, only about 50% is protonated at the physiological pH, and the overall charge density is low, which leads to insufficient transfection efficiency. Therefore, in view of the shortcomings of these existing nucleic acid carriers, it is particularly important to design a nucleic acid carrier with a high transfection efficiency, a good biocompatibility and a low cytotoxicity.

### SUMMARY

In view of the shortcomings of the existing nucleic acid carrier materials, the present disclosure aims to provide a nanoparticle nucleic acid carrier containing hyperbranched polylysine and use thereof.

A nanoparticle nucleic acid carrier containing hyperbranched polylysine is a polycationic gene carrier containing hyperbranched polylysine. The nanoparticle nucleic acid carrier can show a higher transfection rate than that of polyethyleneimine, has a better biocompatibility and a lower cytotoxicity, and can be bonded with the loaded nucleic acid substance through electrostatic action to form stable nanoparticles, so that the nanoparticles are free from enzymatic degradation and immunological recognition clearance, and the purpose of transmembrane transportation is achieved.

The hyperbranched polylysine has a molecular weight of 5000-6000 g/mol.

The loaded nucleic acid substance is at least one of DNA and mRNA.

The stable nanoparticles have a particle size of 16 nm to 156 nm, and the complex has a Zeta potential of −21.53 mV to 17.67 mV and has a high stability.

Preferably, the mass ratio of the hyperbranched polylysine with a molecular weight of 5000-6000 g/mol to the loaded nucleic acid is 1:10 to 5:1.

The present disclosure provides a method for preparing a nanoparticle nucleic acid carrier-loaded nucleic acid complex, including the following steps:
(1) adding hyperbranched polylysine into a phosphate buffer solution, and ultrasonically dissolving;
(2) respectively diluting a nucleic acid substance and the hyperbranched polylysine to a certain concentration by using a phosphate buffer solution as a diluting solvent; and
(3) mixing the obtained nucleic acid substance solution with the hyperbranched polylysine solution, followed by swirling for 10-30 s, and standing for 10-30 min.

Preferably, in steps (1) and (2), the phosphate buffer solution has a concentration of 0.01 mol/L, and a pH of 7.2-7.4.

Preferably, in step (2), the diluted nucleic acid substance solution has a concentration of 1-100 μg/mL, and the diluted hyperbranched polylysine solution has a concentration of 10-500 μg/mL.

In the above technical solution, the nucleic acid takes plasmid DNA containing enhanced green fluorescent protein (eGFP) and eGFP mRNA as models.

The present disclosure further provides the use of the above complex nanoparticles as described in the preparation method, which includes the following steps:
co-culturing the nucleic acid carrier-loaded nucleic acid complex solution with cells to realize the effective transfection of nucleic acid substances in the cells.

In the above technical solution, according to a specific example of the present disclosure, the specific use may be as follows: inoculating cells in a 6-well plate, wherein the density of the inoculated cell suspension is 1×10⁵ cell/mL; after the cells are incubated in a 5% CO₂ incubator for 24 hours, washing the cells with PBS for 2-3 times; adding 800 μL of serum-free medium without double antibodies and 200 μL of the nucleic acid carrier-loaded nucleic acid complex solution to co-culture with the cells for 24 hours.

Preferably, the cells are selected from HEK293, Hela, B16, PC1.0 and Vero cell lines.

The structural formula of the main component, i.e., hyperbranched polylysine, of the nanoparticle nucleic acid carrier containing hyperbranched polylysine is shown in the following figure. Hyperbranched polylysine has the advantages of simple preparation process and low biological toxicity. Because there are a lot of amino groups in its molecules, these amino groups will be protonated at the physiological pH, thus neutralizing the negative charges on the surface of nucleic acids, so that large-volume nucleic acid molecules are compressed from extended structures into smaller nucleic acid particles and wrapped therein. Through endocytosis, the transfection complex can input the loaded nucleic acid molecules into the cells, and then further carry out the transcription, translation and expression of nucleic acid in the cells. Compared with the existing standard gene transfection carrier, Polyethyleneimine (PEI), the nucleic acid carrier in this use shows higher transfection efficiency and lower cytotoxicity, and is expected to play a role in gene therapy, nucleic acid vaccine and other fields.

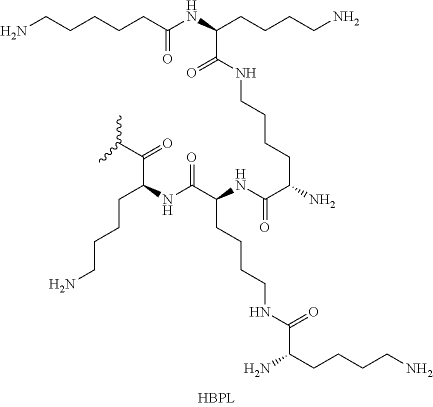 [SMILES: *C(=O)[C@H](CCCCN)NC(=O)[C@H](CCCCNC(=O)[C@@H](N)CCCCN)NC(=O)[C@@H](N)CCCCNC(=O)[C@H](CCCCN)NC(=O)CCCCCN]

The present disclosure has the beneficial effects that.
(1) the preparation method of the nanoparticle nucleic acid carrier containing hyperbranched polylysine provided by the present disclosure is simple, the nucleic acid carrier-loaded nucleic acid complex nanoparticles have a good stability, and both the preparation method and use are convenient and easy to implement with a high repeatability;
(2) the nanoparticle nucleic acid carrier containing hyperbranched polylysine provided by the present disclosure is safe and effective, and has low cytotoxicity; it can be successfully bonded with nucleic acid substances and protect them from degradation by intracellular enzymes, so that the loaded nucleic acid shows a high transfection rate and a high fluorescence expression efficiency in cells.

### BRIEF DESCRIPTION OF DRAWINGS

FIG. 1 shows the (a) number average particle size and (b)Zeta potential of HBPL-DNA nanoparticles with different mass ratios prepared by the present disclosure.

FIG. 2 is the flow single parameter histogram and cell average fluorescence intensity of HBPL-DNA nanoparticles with different mass ratios prepared by this disclosure.

FIG. 3 shows the transfection efficiency of HBPL-DNA nanoparticles with different mass ratios prepared by the present disclosure.

FIG. 4 is a comparison of transfection rates of HBPL-DNA nanoparticles and PEI-DNA in an example of the present disclosure when the mass ratio provided is 3:1.

FIG. 5 shows (a) number average particle size and (b)Zeta potential of HBPL-mRNA nanoparticles with different mass ratios prepared by the present disclosure.

FIG. 6 is the flow single parameter histogram and cell average fluorescence intensity of HBPL-mRNA nanoparticles with different mass ratios prepared by this disclosure.

FIG. 7 shows the transfection efficiency of HBPL-mRNA nanoparticles with different mass ratios prepared by the present disclosure.

FIG. 8 shows the fluorescence images of 293T cells, CHO-K1 cells, HCT116 cells and HeLa cells transfected with hyperbranched polylysine-loaded DNA, in which the scale is 100 μm.

### DETAILED DESCRIPTION OF EMBODIMENTS

In order to introduce the nanoparticle nucleic acid carrier containing hyperbranched polylysine and the use thereof in detail, description will be made below with reference to specific examples.

### Example 1

Preparation of the solution of a nanoparticle nucleic acid carrier containing hyperbranched polylysine: HBPL solutions with concentrations of 10 μg/mL, 50 μg/mL, 100 μg/mL, 250 μg/mL and 500 μg/mL were prepared respectively. The solvent used was a phosphate buffer solution with a concentration of 0.01 mol/L and a pH of 7.2-7.4, and the hyperbranched polylysine was fully dissolved in the solution by ultrasound. When the molecular weight of HBPL is too low, HBPL cannot effectively encapsulate nucleic acid, while when the molecular weight is too high, its cytotoxicity will increase, therefore it is preferable that the molecular weight of HBPL used is 5000-6000 g/mol.

Preparation of a solution containing DNA: a plasmid DNA solution containing enhanced green fluorescent protein (eGFP) was prepared. The solvent used was a phosphate buffer solution with a concentration of 0.01 mol/L and a pH of 7.2-7.4, so that the concentration of nucleic acid solution was 100 μg/mL.

The nucleic acid carrier solution was mixed with the eGFP-DNA solution to prepare a HBPL-DNA complex nanoparticle solution, which was then swirled for 10-30 s, and allowed to stand for 10-30 min.

The particle size and Zeta potential of the complex nanoparticles were measured by a nanoparticle size potentiometer. As shown in FIG. 1(a), the number average particle size of the HBPL-DNA complex nanoparticles with different mass ratios is between 24 nm and 156 nm; the Zeta potentials of the HBPL-DNA complex nanoparticles in FIG. 1(b) are all positive and show an increasing trend with the mass ratio. This is mainly because the positive charge of protonated amino groups in hyperbranched polylysine neutralizes the negative charge on the DNA surface, which makes it aggregate and increases the charge density on the polymer surface, showing that the Zeta potential rises from 1.63 mV to 16.97 mV.

The transfection of intracellular DNA was realized by co-culture of Hela cells with the HBPL-DNA complex solution. The specific steps are as follows: cells were inoculated in a 6-well plate, with the density of the suspension of the inoculated cells being 1×10⁵ cells/mL. After the cells were incubated in a 5% CO₂ incubator for 24 hours, the original complete culture medium in the well plate was removed, followed by washing with PBS for 2 to 3 times, and 800 μL of an RPMI 1640 culture medium and 200 μL of the HBPL-DNA complex solution were added again to co-culture with the cells for 24 hours.

The transfection effect of intracellular DNA was evaluated by cell transfection rate and transfection efficiency.

### a) Determination of Cell Transfection Rate

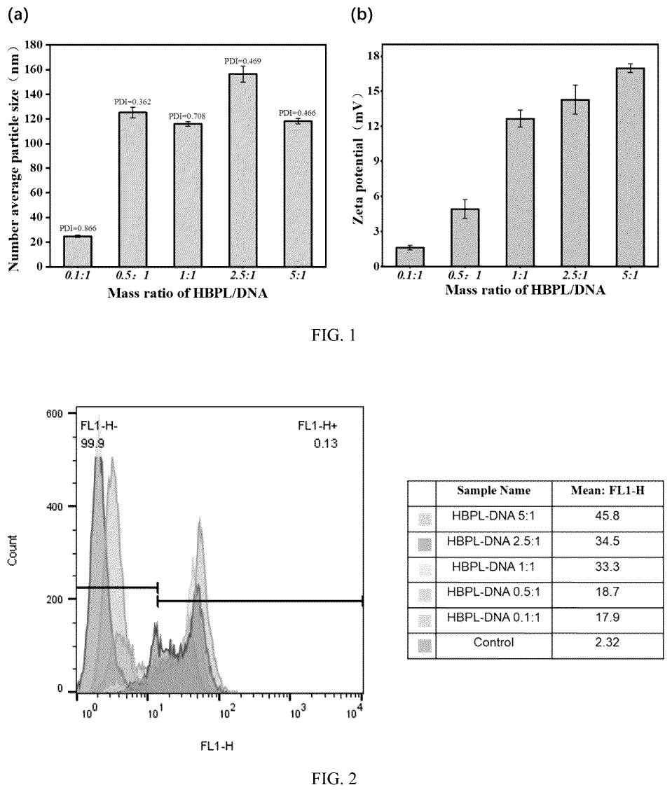

The cell culture plate was taken out from the incubator, the cell culture solution was removed, and the plate was washed twice with a PBS solution; after adding 100-300 μL of trypsin to digest the cells for 2-4 min, 1 mL of a RPMI 1640 medium containing 10% fetal bovine serum was added to stop digestion. The cell fluid in the well plate was transferred to a centrifuge tube in the dark, which was centrifuged at 1000 r/min for 2-4 min, the supernatant was discarded, 500-1000 μL of a PBS solution was added to resuspend the cells, and the cell transfection rate was measured by flow cytometry. The result is shown in FIG. 2. HBPL-DNA complexes with different ratios all showed obvious transfection behavior, and the transfection efficiency of the nanoparticle nucleic acid carrier increased with the increase of the mass ratio of HBPL to DNA.

b) Expression of Enhanced Green Fluorescent Protein (eGFP)

The fluorescence intensity of transfected positive cells was detected by flow cytometry.

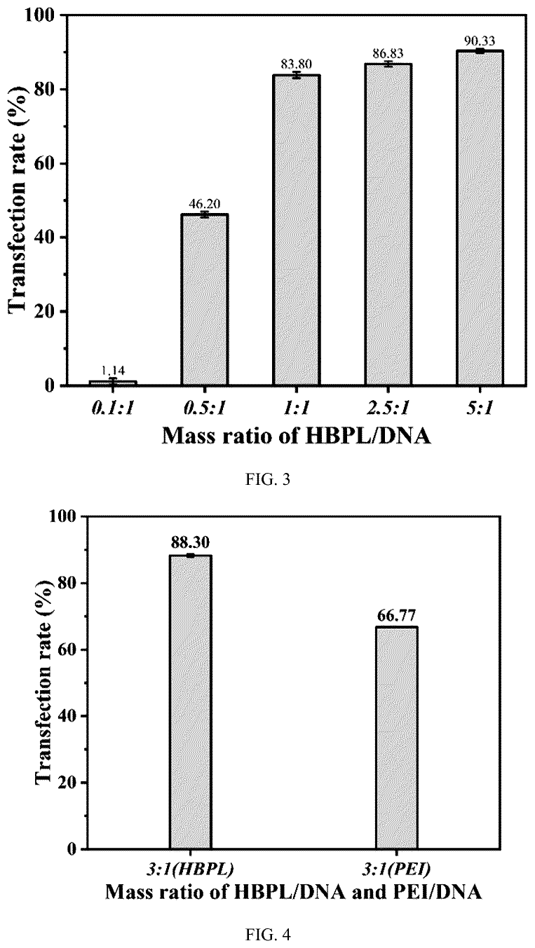

As shown in FIG. 2, compared with the control group, the HBPL-DNA complex nanoparticles with different mass ratios showed obvious fluorescence expression, and the fluorescence intensity increased with the increase of mass ratio: when the mass ratio of HBPL-DNA was 5:1, the transfection rate was as high as 90.33%; FIG. 3 shows the transfection rates corresponding to different HBPL-DNA mass ratios. When the HBPL-DNA mass ratio reached 1:1, the transfection rate of the nanoparticle nucleic acid carrier was as high as 83.80%, which was significantly higher than the transfection rate of polyethyleneimine (66.77%, as shown in FIG. 4) at the same mass ratio, which proves that the nanoparticle nucleic acid carrier containing hyperbranched polylysine proposed by the present disclosure can successfully bind to nucleic acid substances, protect them from intracellular enzyme degradation, so that the loaded nucleic acid shows a higher transfection rate, and a higher fluorescence expression efficiency can also be achieved in cells.

Because enhanced green fluorescent protein (eGFP) can emit high-intensity fluorescence, it is suitable to be used as a reporter gene for study of gene expression, regulation, cell differentiation and the location and transport of proteins in organisms, therefore in the present disclosure, the DNA preferably contains plasmid DNA of enhanced green fluorescent protein (eGFP), but it is not limited to this type of DNA.

### Example 2

The implementation steps were the same as in Example 1 except that the loaded nucleic acid substance was eGFP mRNA, and the system could still successfully achieve effective transfection of mRNA.

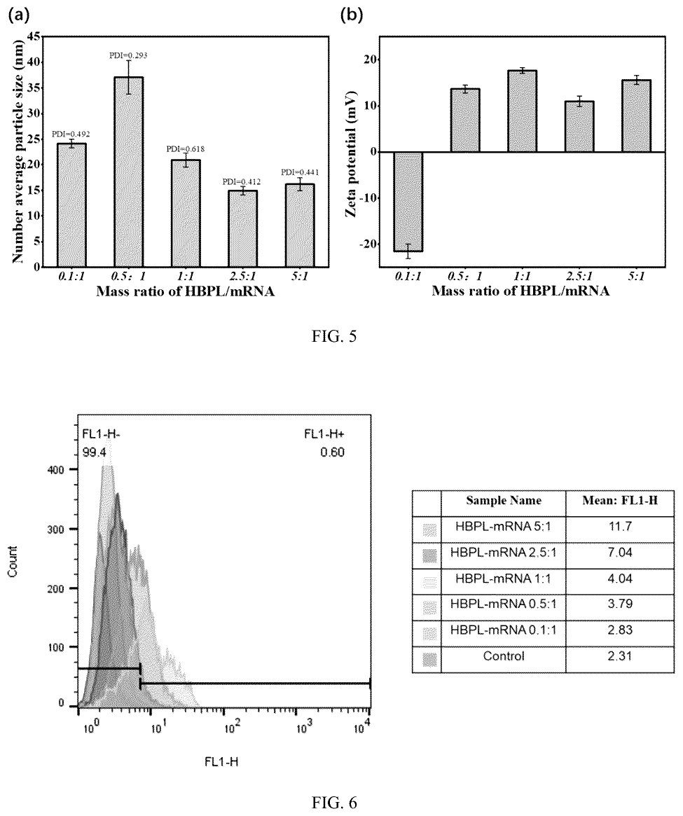

The particle size and Zeta potential of the HBPL-mRNA complex nanoparticles were measured by a nanoparticle size potentiometer. As shown in FIG. 5(a), the number average particle size of HBPL-mRNA complex nanoparticles with different mass ratios was between 14.91 nm-37.06 nm. In FIG. 5(b), when the mass ratio of HBPL-mRNA was 0.5:1, the Zeta potential of the complex nanoparticles changed to a positive value, which was mainly because the positively charged hyperbranched polylysine is bonded to the negatively charged mRNA surface, neutralizing part of the negative charge on the mRNA surface and causing it to aggregate. With the increase of the mass ratio, the surface potential of the polymer was stable at 10.99 mV-17.67 mV, which indicates that HBPL has a good mRNA binding ability under the appropriate mass ratio.

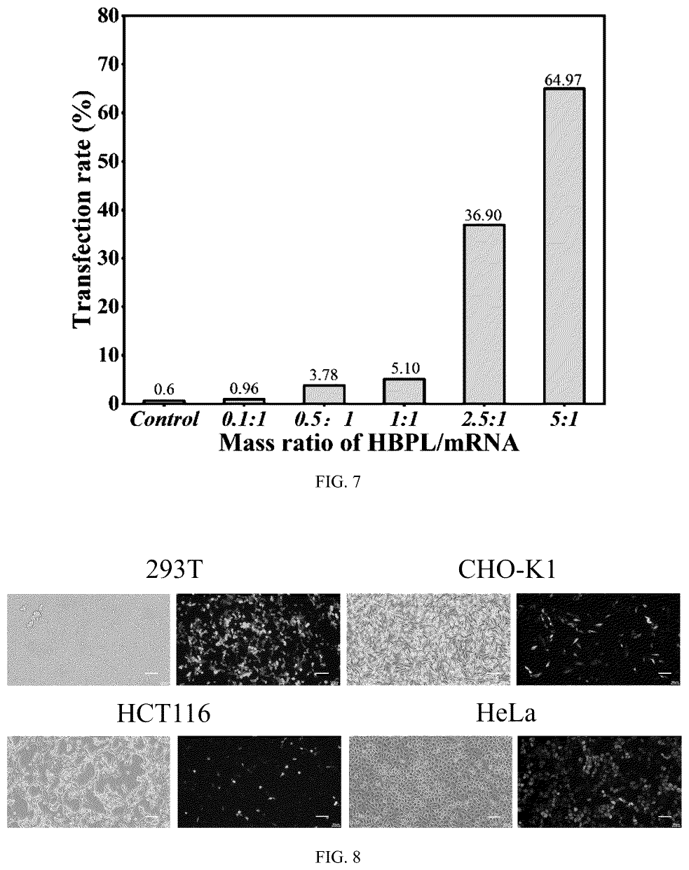

The transfection of mRNA was characterized by flow cytometry. As shown in FIGS. 6 and 7, with the increase of the mass ratio of HBPL-mRNA, the percentage of transfected positive cells and the fluorescence intensity both increased continuously. When the mass ratio of HBPL-mRNA was 5:1, the transfection rate reached 64.97%, which proves that the nanoparticle nucleic acid carrier containing hyperbranched polylysine provided by the present disclosure can effectively transfect mRNA and promote its successful expression in cells to some extent.

In the present disclosure, the mRNA is preferably eGFP mRNA, but not limited to this type of mRNA.

The nanoparticle nucleic acid carrier containing hyperbranched polylysine provided by the present disclosure has a good cell compatibility, which is detected by CCK-8 (Cell Counting Kit-8) method, and the specific implementation steps are as follows:

Cells were inoculated in a 96-well plate, and the density of the suspension of the inoculated cell was 0.5×10⁴ cells/mL. After the cells were incubated in CO₂ incubator for 24 hours, the original complete medium in the well plate was removed. 100 μL of a fresh culture medium and 10 μL of HBPL solutions with different concentrations (10 μg/mL, 50 μg/mL, 100 μg/mL, 250 μg/mL, 500 μg/mL) were added to the experimental group, and 100 μL of a fresh culture medium and 10 μL of a phosphate buffer solution were added to the blank group and then the blank group continued to be cultured for 72 h. The fresh culture medium was replaced, 10 μL of a CCK-8 reagent was added to each well to be incubated in CO₂ incubator for 30 min, and the absorbance (OD value) was measured with enzyme-linked immunosorbent assay at a wavelength of 450 nm. The cell viability is calculated by the following formula:

$$Cell Viability = \frac{O {D}_{t r e a t e d} - O {D}_{blank}}{O {D}_{control} - O {D}_{blank}} \times 100 \%$$

According to the test results of the enzyme-linked immunosorbent assay, the cell compatibility of the hyperbranched polylysine in the concentration range specified in the present disclosure is all above 99.60%, indicating that the nanoparticle nucleic acid carrier containing hyperbranched polylysine provided by the present disclosure has a low cytotoxicity.

### Example 3

The implementation steps were the same as in Example 1, except that the transfected cells were 293T cells, CHO-K1 cells, HCT116 cells and HeLa cells; the mass ratio of HBPL/DNA used was 10:1.

The transfection effect of HBPL/DNA for different cell lines was characterized by a fluorescence microscope. As shown in FIG. 8, HBPL/DNA can deliver DNA to 293T cells, CHO-K1 cells, HCT116 cells and HeLa cells and express the function of DNA.

The embodiments described in this specification are only examples of the realization forms of the present disclosure, and the scope of protection of the present disclosure shall not be regarded as limited to the specific forms stated in the embodiments.

What is claimed is:

1. A nanoparticle nucleic acid carrier containing hyperbranched polylysine, wherein the carrier is a polycationic gene carrier containing hyperbranched polylysine.

2. The nanoparticle nucleic acid carrier containing hyperbranched polylysine according to claim 1, wherein the hyperbranched polylysine has a molecular weight of 5000 g/mol to 6000 g/mol.

3. The nanoparticle nucleic acid carrier containing hyperbranched polylysine according to claim 1, wherein the polycationic gene carrier is at a physiological pH; amino group in the hyperbranched polylysine is protonated, thereby neutralizing negative charge on the surface of nucleic acid, so that a large-volume nucleic acid molecule is concentrated from an extended structure into a small-volume nucleic acid particle and wrapped therein, thereby forming a nucleic acid carrier-loaded nucleic acid complex.

4. The nanoparticle nucleic acid carrier containing hyperbranched polylysine according to claim 3, wherein the nucleic acid is at least one of DNA and mRNA.

5. The nanoparticle nucleic acid carrier containing hyperbranched polylysine according to claim 3, wherein a mass ratio of the hyperbranched polylysine to the loaded nucleic acid in the complex is 1:10 to 5:1, the complex has a particle size of 16 nm to 156 nm and a Zeta potential of −21.53 mV to 17.67 mV.

6. A method for preparing a nanoparticle nucleic acid carrier-loaded nucleic acid complex, comprising:
(1) adding hyperbranched polylysine into a phosphate buffer solution, and ultrasonically dissolving;
(2) respectively diluting a nucleic acid substance and the hyperbranched polylysine to a certain concentrations by using a phosphate buffer solution as a diluting solvent; and
(3) mixing the obtained nucleic acid substance solution with the hyperbranched polylysine solution, swirling for 10 s to 30 s, and standing for 10 min to 30 min.

7. The method for preparing a complex according to claim 6, wherein in steps (1) and (2), the phosphate buffer solution has a concentration of 0.01 mol/L, and a pH of 7.2-7.4.

8. The method for preparing a complex according to claim 6, wherein in step (2), the diluted nucleic acid substance solution has a concentration of 1 μg/mL to 100 μg/mL, and the diluted hyperbranched polylysine solution has a concentration of 10 μg/mL to 500 μg/mL.

9. Use of the complex prepared by the method according to any one of claim 6 in transfection of a nucleic acid substance.

10. The use according to claim 9, wherein effective transfection of the nucleic acid substance in a cell is realized by a co-culturing solution of the nucleic acid carrier-loaded nucleic acid complex solution with a cell selected from HEK293, Hela, B16, PC1.0 and Vero cell lines.
